(19) United States
(12) Patent Application Publication
(10) Pub. No.: US 2026/0177575 A1
(43) Pub. Date: Jun. 25, 2026

(54) ANALYSIS APPARATUS

(71) Applicant: FUJIFILM CORPORATION, Tokyo (JP)

(72) Inventor: Atsushi TAKAHASHI, Kanagawa (JP)

(21) Appl. No.: 19/539,024

(22) Filed: Feb. 13, 2026

Related U.S. Application Data
(63) Continuation of application No. PCT/JP2024/027823, filed on Aug. 2, 2024.

(30) Foreign Application Priority Data
Aug. 30, 2023 (JP) 2023-140486

(51) Int. Cl. G01N 35/02; A61B 5/145; B01L 3/00

(52) U.S. Cl. CPC G01N 35/025; A61B 5/14532; B01L 3/5027; B01L 3/502715

(57) ABSTRACT

An analysis apparatus in which a flat plate-shaped analysis chip onto which a test substance sample is spotted is detachably loaded is configured to analyze the test substance sample by using the analysis chip, the analysis apparatus including a transportation table on which the analysis chip is transported, and a transportation mechanism that transports the analysis chip positioned at a standby position on the transportation table to a predetermined target position while sliding the analysis chip on the transportation table by pressing the analysis chip from a rear end, the transportation mechanism including a plate-shaped pressing member configured to press the analysis chip toward the target position by pressing the rear end with a tip thereof, and a cover member capable of covering a portion of an upper surface of the analysis chip on a side of the rear end while the tip presses the rear end.

### CROSS-REFERENCE TO RELATED APPLICATIONS

This application is a continuation of International Application No. PCT/JP2024/027823, filed on August 2, 2024, which claims priority from Japanese Patent Application No. 2023-140486, filed on August 30, 2023. The entire disclosure of each of the above applications is incorporated herein by reference.

### BACKGROUND

### 1. Technical Field

The present disclosure relates to an analysis apparatus.

### 2. Related Art

An analysis apparatus that analyzes a test substance sample by using an analysis chip onto which the test substance sample is spotted is known. As analysis of the test substance sample, for example, the concentration of a test target substance included in the test substance sample is measured by measuring a state of a reaction between the test substance sample and a reagent. The test substance sample is, for example, blood, urine, or the like. In one example, the analysis chip is a dry analysis chip including a reagent layer that includes a reagent. The analysis chip is also referred to as a measurement element or a chemical analysis slide.

The analysis apparatus includes a spotting mechanism that spots a test substance sample onto an analysis chip, and an incubator that heats a plurality of analysis chips to ensure suitable measurement conditions.

In the analysis apparatus disclosed in JP1990-184761A (JP-H2-184761A), a spotting position onto which a test substance sample is spotted onto an analysis chip is provided outside the incubator, and a slide cartridge that houses a plurality of analysis chips is disposed in the vicinity of the spotting position. This analysis apparatus includes a slide supply mechanism that discharges slides (corresponding to analysis chips) one by one from inside the slide cartridge, and slide insertion/discharge means that transports each of the slides discharged from the slide supply mechanism to the spotting position and that inserts the slides into respective cells of the incubator.

### SUMMARY

In the analysis apparatus in JP1990-184761A (JP-H2-184761A), the slide supply mechanism includes a plate-shaped member (corresponding to a pressing member) and is configured to push out a slide through a slide-discharge slit by inserting the plate-shaped member into a discharge-means entrance slit included in the cartridge and by pushing the rear end of the slide with the plate-shaped member. In JP1990-184761A (JP-H2-184761A), the plate-shaped member with which the slide is pushed pushes the slide in a state in which the plate-shaped member is in contact with only a portion of the rear end of the slide. Therefore, the orientation of the slide during transportation thereof tends to be unstable. For example, the orientation of the slide may incline relative to an advancing direction, or the slide may lift up from a transportation table. When the orientation of the slide is unstable during transportation thereof, malfunction such as a transportation failure is likely to occur during transportation of the slide.

The present disclosure has been made in view of the above circumstances, and an object of the present disclosure is to provide an analysis apparatus including a transportation mechanism in which occurrence of malfunction during transportation of an analysis chip is suppressed.

The analysis apparatus according to the present disclosure is an analysis apparatus in which an analysis chip having a flat plate shape and onto which a test substance sample is spotted is detachably loaded, the analysis apparatus being configured to analyze the test substance sample by using the analysis chip, the analysis apparatus including:

a transportation table on which the analysis chip is transported; and

a transportation mechanism that transports the analysis chip positioned at a standby position on the transportation table to a target position that is predetermined, while sliding the analysis chip on the transportation table by pressing a rear end of the analysis chip,

in which the transportation mechanism includes a pressing member and a cover member, the pressing member having a plate shape and being configured to press the analysis chip toward the target position by pressing the rear end of the analysis chip with a tip of the pressing member, the cover member being capable of covering, in a state of protruding forward in a transportation direction beyond the tip of the pressing member, a portion of an upper surface of the analysis chip on a side of the rear end while the tip of the pressing member presses the rear end of the analysis chip.

In the analysis apparatus of the present disclosure, at least one of the transportation table or the cover member may include a guide portion that positions the analysis chip in a width direction.

The analysis apparatus of the present disclosure may include a loading unit provided with a cell in which the analysis chip is to be housed for measurement, and the target position may be a position at which the cell is provided in the loading unit.

The loading unit may be an incubator that heats the analysis chip housed in the cell to a predetermined temperature.

In the analysis apparatus of the present disclosure, the cover member is preferably disposed to overlap the pressing member so as to be slidable along the transportation direction, and the analysis apparatus preferably further includes a contact member that comes into contact with the cover member at a predetermined position before the analysis chip reaches the target position, the contact member being configured to restrict an advance of the cover member to the target position and thereby retract the cover member from a protruding position at which the cover member protrudes forward in the transportation direction beyond the tip of the pressing member.

In the analysis apparatus of the present disclosure, the analysis apparatus preferably has an urging member that urges the cover member toward the protruding position, and it is preferable that the cover member retracts relative to the pressing member against an urging force of the urging member when coming into contact with the contact member and returns to the protruding position when separating from the contact member.

The analysis apparatus of the present disclosure may include a loading unit provided with a cell in which the analysis chip is to be housed for measurement, the target position may be a position at which the cell is provided in the loading unit, an opening frame having a slit through which the analysis chip passes may be provided in a path for insertion into the cell, and the contact member may be the opening frame.

The analysis apparatus according to the present disclosure may include a spotting mechanism that spots the test substance sample onto the analysis chip, and the target position may be a spotting position onto which the test substance sample is spotted.

The analysis chip may includes a spotting portion on a main surface of the analysis chip, and, in the analysis apparatus, the cover member may cover the upper surface in a state in which the spotting portion of the analysis chip is exposed.

In the analysis apparatus of the present disclosure, the transportation table may have an opening at the standby position, the analysis apparatus may include a cartridge installation portion at which a cartridge that houses a plurality of the analysis chips that are stacked is detachably installed, the cartridge installation portion being configured to hold the cartridge below the standby position on the transportation table, and the cartridge may include a push-up mechanism that pushes up, among the plurality of analysis chips housed in the cartridge, an uppermost analysis chip through the opening to a height at which a lower surface of the uppermost analysis chip coincides with a transportation surface of the transportation table.

According to the technology of the present disclosure, it is possible to provide an analysis apparatus including a transportation mechanism in which occurrence of malfunction during transportation of an analysis chip is suppressed.

### BRIEF DESCRIPTION OF THE DRAWINGS

FIG. 1 is a schematic diagram illustrating an overall configuration of an analysis apparatus according to an embodiment;

FIG. 2 is a plan view of a part of the analysis apparatus;

FIG. 3 is a sectional view of a transportation path section for an analysis chip in the analysis apparatus;

FIG. 4 is an external perspective view illustrating an example of the structure of an analysis chip;

FIG. 5 illustrates a state of transportation of an analysis chip;

FIG. 6 is a perspective view of a part of the transportation mechanism as viewed from the side of a cover member;

FIG. 7 is a perspective view of the part of the transportation mechanism as viewed from the side of a pressing member;

FIG. 8 is a perspective view illustrating a state in which an analysis chip is held by the transportation mechanism;

FIG. 9A illustrates an analysis chip immediately before being loaded in a cell, and FIG. 9B illustrates an analysis chip loaded in a cell;

FIGS. 10A and 10B illustrate a state of sliding between the pressing member and the cover member;

FIGS. 11A and 11B illustrate transportation of an analysis chip in the analysis apparatus;

FIGS. 12A and 12B illustrate transportation of an analysis chip in the analysis apparatus;

FIG. 13 illustrates a configuration in which an analysis chip is disposed at a standby position on a transportation table by using a cartridge that houses the analysis chip; and

FIG. 14 illustrates a state of transportation of an analysis chip.

### DESCRIPTION OF EMBODIMENTS

Hereinafter, a preferred embodiment of the present disclosure will be described with reference to the drawings.

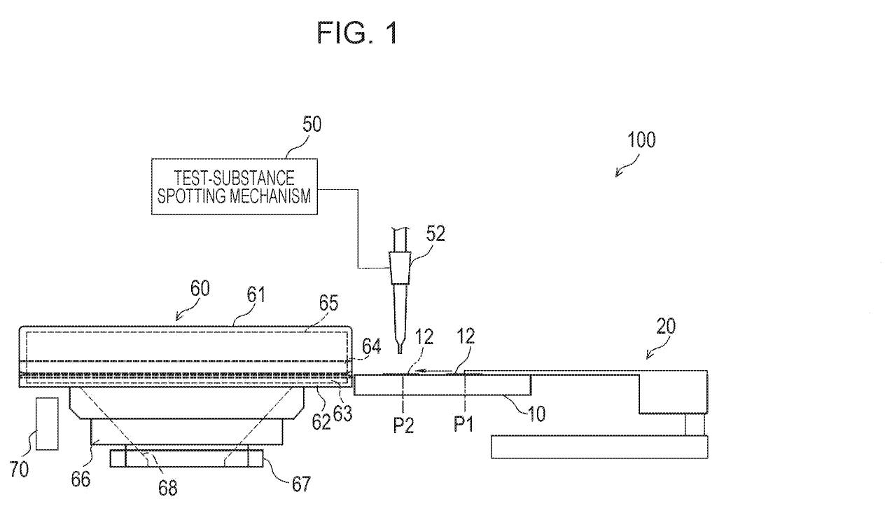

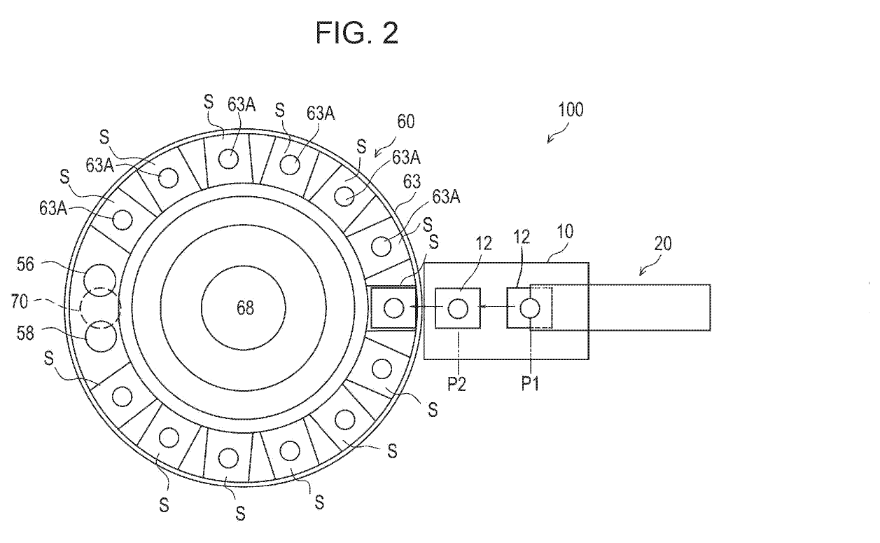

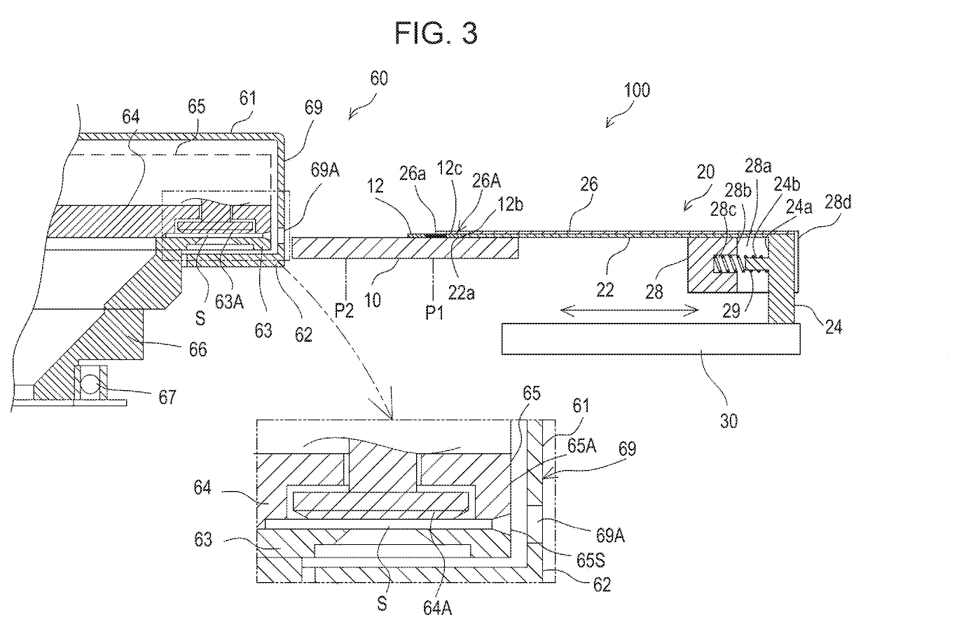

FIG. 1 is a schematic diagram illustrating an overall configuration of an analysis apparatus 100 according to an embodiment, and FIG. 2 is a plan view of a principal part of the analysis apparatus 100 in FIG. 1. FIG. 3 is a sectional view of a transportation path section for an analysis chip 12 in the analysis apparatus 100.

The analysis apparatus 100 is an analysis apparatus that analyzes a test substance sample. The analysis apparatus 100, for example, uses an analysis chip 12 of a dry type to measure the concentration of a test target substance included in the test substance sample. When the analysis chip 12 has a flat plate shape, the analysis chip 12 is also referred to as, for example, a slide. The analysis apparatus 100 is an example of "analysis apparatus" according to the technology of the present disclosure.

Specifically, in the analysis apparatus 100 in the present example, blood is used as a test substance sample and the concentration of a test target substance included in the blood is optically measured. More specifically, the concentration of the test target substance is measured by colorimetry measurement.

As illustrated in FIG. 1, the analysis apparatus 100 includes a transportation table 10, a transportation mechanism 20, a test-substance spotting mechanism 50, an incubator 60, and an optical measurement unit 70.

First, the analysis chip 12 to be used for analysis will be briefly described, and then, a configuration of the analysis apparatus 100 will be described.

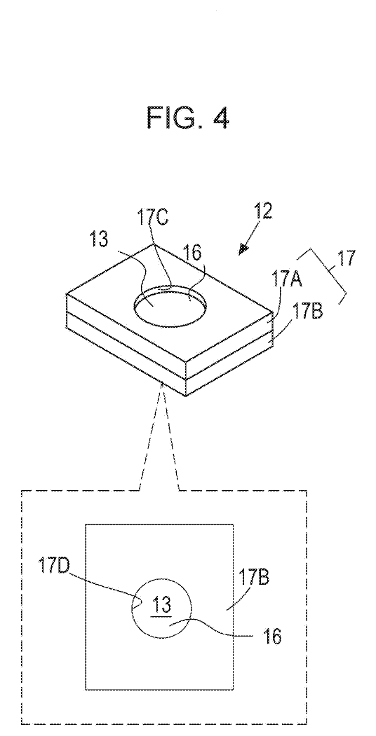

FIG. 4 is an external perspective view illustrating an example of the structure of the analysis chip 12. The analysis chip 12 used in the analysis apparatus 100 is an object to be transported from the transportation table 10 to the incubator 60 by the transportation mechanism 20.

As illustrated in FIG. 4, the analysis chip 12 has a reaction region 13 in which a reagent is immobilized. The reagent reacts with a test target substance and thereby generates a substance that develops a specific color. The substance that develops the color due to this reaction is hereinafter referred to as the reactant. As the reagent, for example, a solid-phase dry reagent that is in a dry state at least at the time of shipment is used. The test substance sample is spotted onto the reaction region 13 of the analysis chip 12.

The analysis chip 12 has a carrier 16 onto which a test substance sample is spotted, and the carrier 16 is housed in a case 17. The case 17 is constituted by a first case 17A and a second case 17B, and the first case 17A and the second case 17B houses the carrier 16 therebetween. The first case 17A has an opening 17C functioning as a drip port for spotting a test substance sample onto the reaction region 13. The second case 17B has an opening 17D for irradiating the reaction region 13 with light. The carrier 16 is exposed in the opening 17C of the first case 17A constituting a surface of the analysis chip 12. The carrier 16 is also exposed in the opening 17D of the second case 17B constituting a back surface of the analysis chip 12. A region exposed in the opening 17C and a region exposed in the opening 17D constitute the reaction region 13 in which a reagent is immobilized.

A test substance sample is dropped onto the reaction region 13 of the analysis chip 12 having the above-described configuration, the analysis chip 12 on which the test substance sample has been dropped is transported into the incubator 60, and the test substance sample is analyzed.

The transportation table 10 is a table for transporting the analysis chip 12 to the incubator 60. As illustrated in FIGS. 1 and 2, the analysis chip 12 before being set in the incubator 60 is placed on the transportation table 10. The analysis chip 12 is transported by sliding on the transportation table 10. A standby position P1 at which the analysis chip 12 is put on standby and a spotting position P2 at which a test substance is spotted onto the analysis chip 12 are provided on the transportation table 10.

The transportation mechanism 20 transports the analysis chip 12 positioned at the standby position P1 on the transportation table 10 to the spotting position P2 and further transports the analysis chip 12 to a cell S (see FIG. 3) of the incubator 60. The transportation mechanism 20 transports the analysis chip 12 by pressing the analysis chip 12 from the rear end 12b such that the analysis chip 12 slides on the transportation table 10. Each of the spotting position P2 and the cell S is an example of "target position that is predetermined" in the technology of the present disclosure. Details of the transportation mechanism 20 will be described later.

The test-substance spotting mechanism 50 spots a test substance sample, such as blood plasma, whole blood, blood serum, or urine, onto the analysis chip 12 that has been transported to the spotting position P2. The test-substance spotting mechanism 50 includes a nozzle 52, an aspiration-discharge mechanism (not illustrated), and a moving mechanism that moves the nozzle 52. The test-substance spotting mechanism 50 aspirates a test substance sample from a test-substance housing portion (not illustrated) and spots the test substance sample onto the analysis chip 12 positioned at the spotting position P2.

A plurality of the analysis chips 12 can be held in the incubator 60. The incubator 60 has a heater inside thereof and has a function of heating each analysis chip 12 in the incubator 60 to a predetermined target temperature and maintaining the analysis chip 12 at the target temperature. More specifically, the incubator 60 maintains an atmosphere around a region onto which a test substance sample is spotted in each analysis chip 12 at the target temperature. The target temperature is, for example, 37°C.

The incubator 60 includes an upper cover 61 and a lower cover 62. Various members constituting the incubator 60 and the analysis chips 12 are housed in a space formed by the upper cover 61 and the lower cover 62. In addition, a rotary cylinder 66 is provided below the lower cover 62. A bearing 67 is disposed below the outer periphery of the rotary cylinder 66, and the rotary cylinder 66 is rotatably supported by the bearing 67. A rotational force is transmitted through the rotary cylinder 66 to the members provided in the incubator 60. The sectional shape of the rotary cylinder 66 is substantially an inverted triangle having an inner diameter decreasing downward. The rotary cylinder 66 has an open bottom that forms a vertex portion of the inverted triangle, and the opening functions as a discarding hole 68 through which each analysis chip 12 that has been used is discarded. Each used analysis chip 12 is moved from a state of being loaded in the cell S toward the center of the rotary table 63 and is dropped toward an inclined surface of the rotary cylinder 66. The used analysis chip 12 that has been dropped into the rotary cylinder 66 slides down the inclined surface to be discarded through the discarding hole 68.

The incubator 60 has, in a space formed between the upper cover 61 and the lower cover 62, a rotating body 65 including a rotary table 63 and a chip pressing member 64. As illustrated in FIG. 2, the rotary table 63 has the cells S, which are a plurality of regions partitioned along a circumferential direction, and each cell S is capable of housing the analysis chip 12. As illustrated in FIG. 3, the chip pressing member 64 has a plurality of projecting portions 64A each at a position facing a corresponding one of the cells S. The projecting portions 64A are urged downward by an urging member (not illustrated). A slit-shaped space is formed between each projecting portion 64A and the corresponding one of the cells S, and the analysis chip 12 is loaded in the space. The projecting portions 64A press, from above, the analysis chips 12 loaded in the cells S. Consequently, a movement of each analysis chip 12 (for example, radially outward displacement of each analysis chip 12 by a centrifugal force when the centrifugal force is generated in the analysis chip 12 due to the rotation of the rotary table 63) in the cell S is suppressed.

Each analysis chip 12 is inserted through an opening portion 69A provided in an outer peripheral wall 69 of the incubator 60 into the incubator 60 and is loaded between the chip pressing member 64 and the cell S of the rotary table 63.

As illustrated in FIG. 2, on the rotary table 63 of the incubator 60 in the present example, a plurality (thirteen) of the cells S are provided along the circumference. The analysis chips 12 placed on the plurality of cells S are sequentially transported to a measurement position by the rotation of the rotary table 63.

An opening window 63A for photometry is formed at a central portion of the bottom surface of each cell S of the rotary table 63, and reflectance optical density of each analysis chip 12 is measured through the opening window 63A by the optical measurement unit 70. A black-density plate 56 and a white-density plate 58 are included at a part of the circumference at which the plurality of cells S are provided. An opening window (not illustrated) is also formed below the black-density plate 56 and the white-density plate 58.

The optical measurement unit 70 is a unit that performs colorimetry measurement, which is optical-density measurement using colorimetry, on the analysis chips 12. The optical measurement unit 70 is provided below the lower cover 62 in an outer peripheral portion of the incubator 60 (see FIG. 1).

The optical measurement unit 70 irradiates each analysis chip 12 with measurement light and receives reflected light from the analysis chip 12, thereby measuring optical density corresponding to a state of a reaction between a test substance sample and a reagent in the analysis chip 12. The black-density plate 56 and the white-density plate 58 are density plates for acquiring reference optical density, which is referred to when optical density of each analysis chip 12 is measured. The optical measurement unit 70 has a plurality of light sources that emit measurement light having a plurality of different wavelengths, and the wavelengths are used depending on the types of the analysis chips 12, in other words, depending on measurement items. Reference optical black-density and reference optical white-density are measured for each wavelength of the measurement light.

The analysis apparatus 100 includes a control unit (not illustrated) that controls operation of the entire apparatus. The control unit is realized by a computer including a processor constituted by a central processing unit (CPU), a non-volatile memory (NVM), a random access memory (RAM), and the like.

Hereinafter, the transportation mechanism 20 will be described in detail. The transportation mechanism 20 transports the slide-shaped analysis chips 12 described with reference to FIG. 4. Specifically, the transportation mechanism 20 transports the analysis chip 12 positioned at the standby position P1 on the transportation table 10 to the spotting position P2 or from the spotting position P2 to one of the cells S of the incubator 60 by pressing the analysis chip 12 from the rear end 12b while sliding the analysis chip 12 on the transportation table 10.

As illustrated in FIG. 3, the transportation mechanism 20 includes a pressing member 22, a cover member 26, and a driving unit 30. The pressing member 22 is a plate-shaped member and presses the analysis chip 12 toward the spotting position P2 or one of the cells S by pressing the rear end 12b of the analysis chip 12 with a tip 22a of the pressing member 22. While the rear end 12b of the analysis chip 12 is pressed by the tip 22a of the pressing member 22, the cover member 26 covers, in a state of protruding forward in a transportation direction beyond the tip 22a of the pressing member 22, a portion of an upper surface 12c of the analysis chip 12 on the side of the rear end 12b. The driving unit 30 moves the pressing member 22 in the transportation direction (the direction of the double-headed arrow in FIG. 3). In the following description, a direction toward the cells S of the incubator 60 is referred to as forward in the transportation direction.

The pressing member 22 has an end portion opposite to the tip 22a with which the rear end 12b of the analysis chip 12 is pressed, and the end portion is fixed to a pressing-member support portion 24. The pressing-member support portion 24 is connected to the driving unit 30 and is moved in the transportation direction by the driving unit 30, thereby moving the pressing member 22 on the transportation table 10 in the transportation direction.

The driving unit 30 is, for example, a linear actuator. A publicly-known configuration can be applied to the linear actuator. The driving unit 30 includes, for example, a motor, pulleys provided at both ends in the transportation direction, and a timing belt wound around the pulleys. The timing belt is directly or indirectly fixed to the pressing-member support portion 24 and moves the pressing member 22 in the transportation direction by rotation of the motor. The driving unit 30 can advance the pressing member 22 in a direction toward the incubator 60 or retract the pressing member 22 in a direction away from the incubator 60 depending on the direction of rotation of the motor.

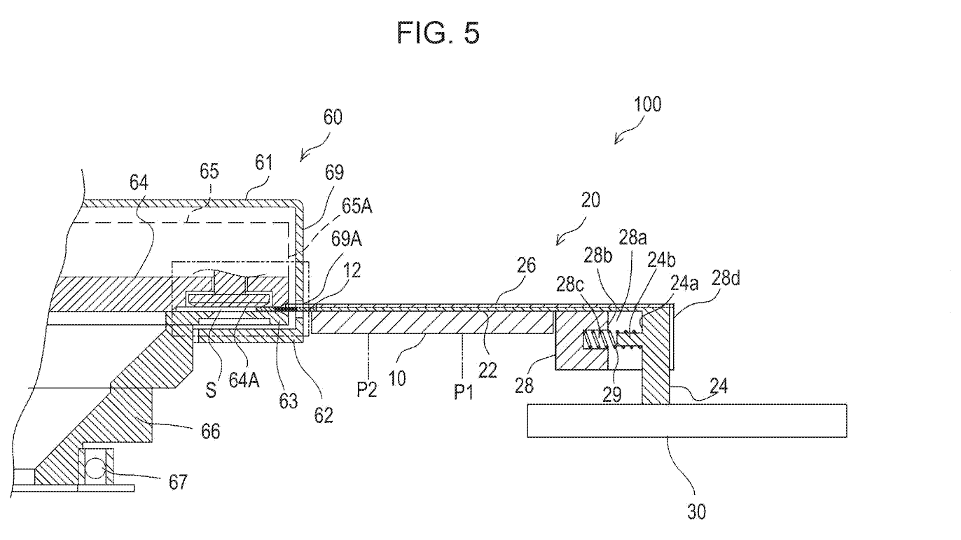

FIG. 5 is a diagram illustrating a state in which the pressing member 22 is advanced toward the incubator 60 by the driving unit 30 in the transportation path illustrated in FIG. 3. As illustrated in FIG. 5, the pressing member 22 and the cover member 26 are integrally moved on the transportation table 10 to thereby transport the analysis chip 12.

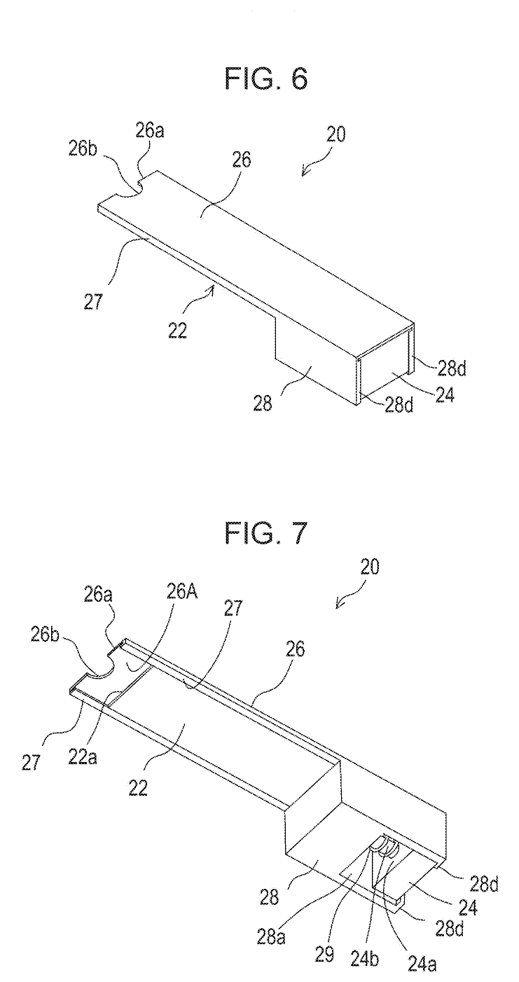

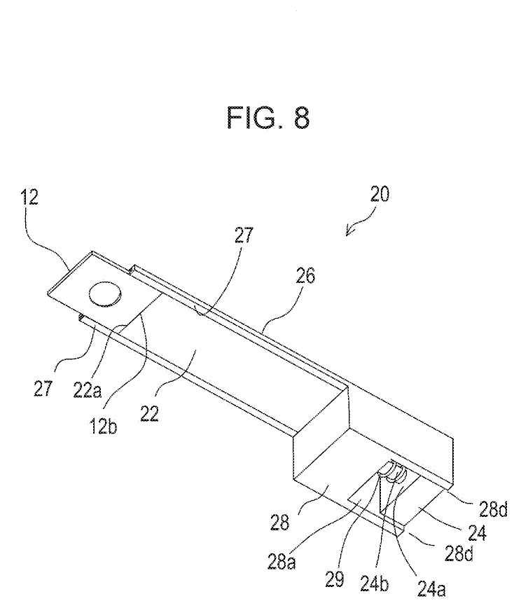

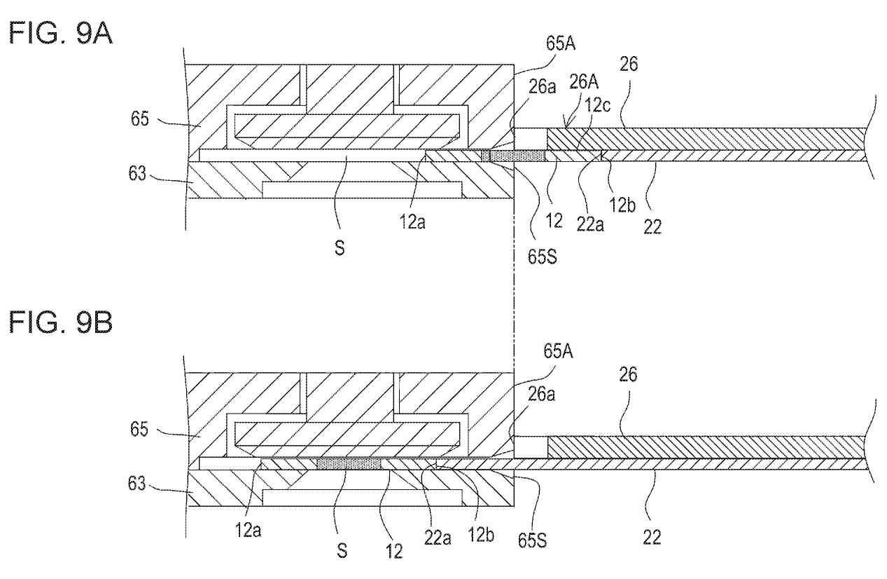

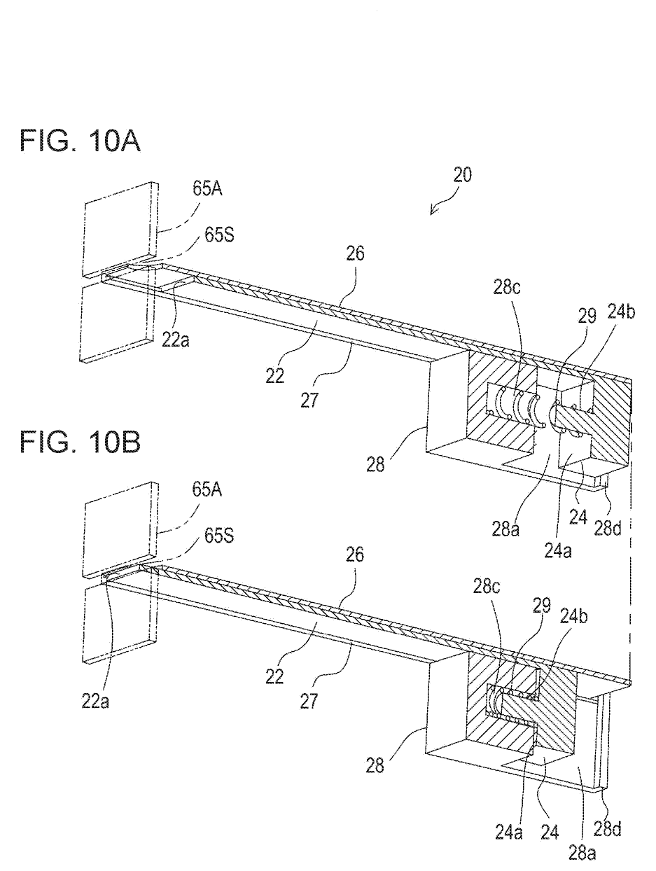

A relationship between the pressing member 22 and the cover member 26 will be described in more detail with reference to FIGS. 6 to 10. FIG. 6 is a perspective view of a main part of the transportation mechanism 20, in which the pressing member 22 and the cover member 26 are stacked, as viewed from the side of the cover member 26, and FIG. 7 is a perspective view thereof as viewed from the side of the pressing member 22. FIG. 8 illustrates a state in which the analysis chip 12 being transported is held by the transportation mechanism 20. FIG. 9A illustrates the analysis chip 12 immediately before being loaded in the cell S, and FIG. 9B illustrates the analysis chip 12 loaded in the cell S. FIGS. 10A and 10B are explanatory perspective views of a state of sliding between the pressing member 22 and the cover member 26.

As illustrated in FIG. 3 mentioned above, the cover member 26 is disposed to overlap the pressing member 22 so as to be slidable along the transportation direction. As illustrated in FIG. 7, engagement portions 27 that engage with two sides of the pressing member 22 in the width direction are provided at two ends of the cover member 26 in the width direction. The pressing member 22 moves on the transportation table 10 in the transportation direction in a state in which the two ends thereof in the width direction are engaged with the engagement portions 27 of the cover member 26. In the present example, a distance between the two engagement portions 27 is substantially equal to the width of the analysis chip 12, and, as illustrated in FIG. 8, the analysis chip 12 is disposed between one engaging portion 27 and the other engaging portion 27 and is transported. That is, the engagement portions 27 also each function as a guide portion for positioning the analysis chip 12 in the width direction. Each engagement portion 27 is an example of "guide portion" in the technology of the present disclosure.

A tip 26a of the cover member 26 is provided with a cutout 26b for exposing the reaction region 13, which is a spotting portion of the analysis chip 12, when the upper surface 12c of the analysis chip 12 is covered.

The cover member 26 has an end portion opposite to a tip portion 26A that covers a portion of the upper surface 12c of the analysis chip 12 on the side of the rear end 12b, and the end portion is fixed to a cover-member support portion 28. The cover-member support portion 28 has a housing portion 28a that houses the pressing-member support portion 24 (see FIGS. 3 and 7). The pressing-member support portion 24 has a substantially rectangular parallelepiped shape and includes, on one surface 24a perpendicular to the transportation direction, a columnar portion 24b protruding in the transportation direction. The housing portion 28a of the cover-member support portion 28 has a surface 28b facing, in a state of housing the pressing-member support portion 24, the one surface 24a of the pressing-member support portion 24, and the surface 28b includes a cylindrical portion 28c that receives the columnar portion 24b provided on the one surface 24a of the pressing-member support portion 24. The pressing-member support portion 24 is movable in the housing portion 28a from a state in which the columnar portion 24b is not inserted into the cylindrical portion 28c to a state in which the columnar portion 24b is inserted into the cylindrical portion 28c (see FIG. 10). A restriction portion 28d that restricts the pressing-member support portion 24 to move to outside the housing portion 28a is provided at the terminal end side of the cover-member support portion 28 in the transportation direction.

A cylindrical coil spring 29 is disposed between the columnar portion 24b and the cylindrical portion 28c. One end of the coil spring 29 is inserted into the cylindrical portion 28c, and the columnar portion 24b is inserted into the coil spiring 29 from the other end side of the coil spring 29. In a state of not being applied with an external force, the surface 28b of the cover-member support portion 28 is maintained to be in a state of being separated at a constant distance from the one surface 24a of the pressing-member support portion 24 due to the urging force of the coil spring 29. As a result, the tip portion 26A of the cover member 26 is able to cover, in a state of protruding to a protruding position forward of the tip 22a of the pressing member 22 in the transportation direction, the upper surface 12c of the side of the rear end 12b of the analysis chip 12. The coil spring 29 is an example of "urging member" of the technology in the present disclosure.

When the pressing-member support portion 24 is moved in the transportation direction by the driving unit 30, the pressing-member support portion 24 and the cover-member support portion 28 move with a constant distance therebetween due to the urging force of the coil spring 29, and thus, the cover-member support portion 28 moves with the movement of the pressing-member support portion 24. In other words, due to the urging force of the coil spring 29, the pressing member 22 and the cover member 26 integrally move in a state in which the tip portion 26A of the cover member 26 protrudes to the protruding position forward of the tip 22a of the pressing member 22 in the transportation direction (see FIGS. 3 and 5).

FIG. 9A is an enlarged view of an area around the cell S immediately before the analysis chip 12 is inserted as illustrated in FIG. 5 into the cell S by being transported in the transportation direction toward the incubator 60. As illustrated in FIG. 9A, in a state in which the tip 26a of the cover member 26 is in contact with an outer peripheral wall 65A of the rotating body 65, a tip 12a of the analysis chip 12 is inserted into the slit 65S of the outer peripheral wall 65A of the rotating body 65. The forward movement of the cover member 26 is restricted by the outer peripheral wall 65A, and the cover member 26 stops at the outer peripheral wall 65A. Thereafter, as illustrated in FIG. 9B, only the pressing member 22 continues to move forward and transports the analysis chip 12 into the cell S. The incubator 60 is an example of "loading unit" according to the technology of the present disclosure. The outer peripheral wall 65A of the rotating body 65 is an example of a contact member that comes into contact with the cover member 26 at a predetermined position before the analysis chip 12 reaches the cell S, which is the target position. The outer peripheral wall 65A restricts an advance of the cover member 26 to the target position, thereby retracting the cover member 26 from the protruding position at which the cover member 26 protrudes forward in the transportation direction beyond the tip 22a of the pressing member 22. The outer peripheral wall 65A is also an example of an opening frame that is provided in a path for insertion of the analysis chip 12 into the cell S and that has the slit 65S through which the analysis chip 12 passes.

FIG. 10A illustrates a state in which the tip 26a of the cover member 26 is in contact with the outer peripheral wall 65A of the rotating body 65. When the pressing member 22 is further moved forward from this state, the pressing-member support portion 24 is moved relative to the cover member 26, as illustrated in FIG. 10B, against the urging force of the coil spring 29. As the pressing-member support portion 24 is moved forward in the transportation direction by the driving unit 30, the coil spring 29 is compressed between the inside of the cylindrical portion 28c and the one surface 24a of the pressing-member support portion 24. Consequently, only the pressing member 22 slides and moves forward relative to the cover member 26 in a state in which the forward movement of the cover member 26 is restricted. By being pressed by the pressing member 22, the analysis chip 12 is inserted into the rotating body 65 through the slit 65S provided on the outer circumferential side of the rotary table 63 and the chip pressing member 64. The tip 22a of the pressing member 22 passes through the slit 65S and presses the analysis chip 12 until the analysis chip 12 is loaded into the cell S.

When the pressing member 22 is retracted rearward in the transportation direction after the analysis chip 12 is loaded into the cell S, the compression of the coil spring 29 is gradually released, and the coil spring 29 returns to a standard length. The movement of the pressing member 22 retracting relative to the cover member 26 by sliding is restricted by the restriction portion 28d of the cover-member support portion 28, and, when the restriction portion 28d of the cover-member support portion 28 comes into contact with the rear end of the pressing-member support portion 24, the cover member 26 and the pressing member 22 retract integrally.

Due to the coil spring 29 as the urging member, the cover member 26 retracts relative to the pressing member 22 against the urging force of the coil spring 29 when the movement of the cover member 26 is restricted by the contact with the contact member (in the present example, the outer peripheral wall 65A of the rotating body 65), and the cover member 26 returns to the protruding position, at which the tip 26a of the cover member 26 protrudes beyond the tip 22a of the pressing member 22, when the cover member 26 is separated from the contact member and restriction of the movement thereof is released.

When the analysis chip 12 is transported to the cell S of the incubator 60, the analysis chip 12 is caused to pass through the opening portion 69A, which is provided in the outer peripheral wall 69 of the incubator 60, and is loaded in the cell S.

A process of analyzing a test substance sample in the analysis apparatus 100 according to the present embodiment will be briefly described.

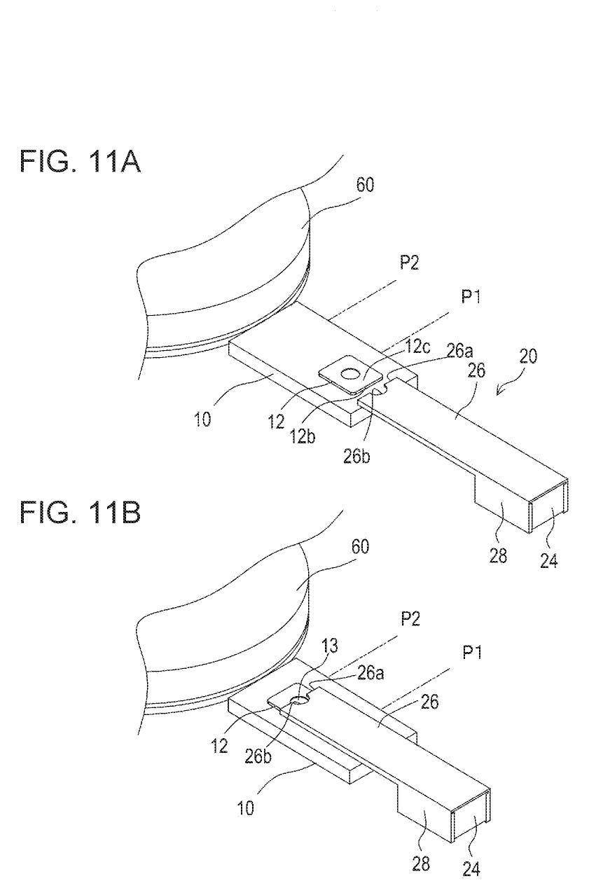

In the analysis apparatus 100, first, as illustrated in FIG. 11A, the analysis chip 12 placed at the standby position P1 is transported to the spotting position P2 as illustrated in FIG. 11B. During transportation, as already described, the analysis chip 12 is transported to the spotting position P2 by the transportation mechanism 20 in a state in which the rear end 12b is pressed by the tip 22a of the pressing member 22 and in which a portion of the upper surface 12c of the analysis chip 12 on the side of the rear end 12b is covered by the tip portion 26A of the cover member 26. The test-substance spotting mechanism 50 spots a test substance sample onto the spotting position P2. As illustrated in FIGS. 11A and 11B, the tip 26a of the cover member 26 has the cutout 26b, and the reaction region 13 of the analysis chip 12 is exposed through the cutout 26b. Consequently, a test substance sample is spotted onto the reaction region 13 in the same state as the state thereof during transportation, in which the analysis chip 12 is urged against the transportation table 10 by the cover member 26.

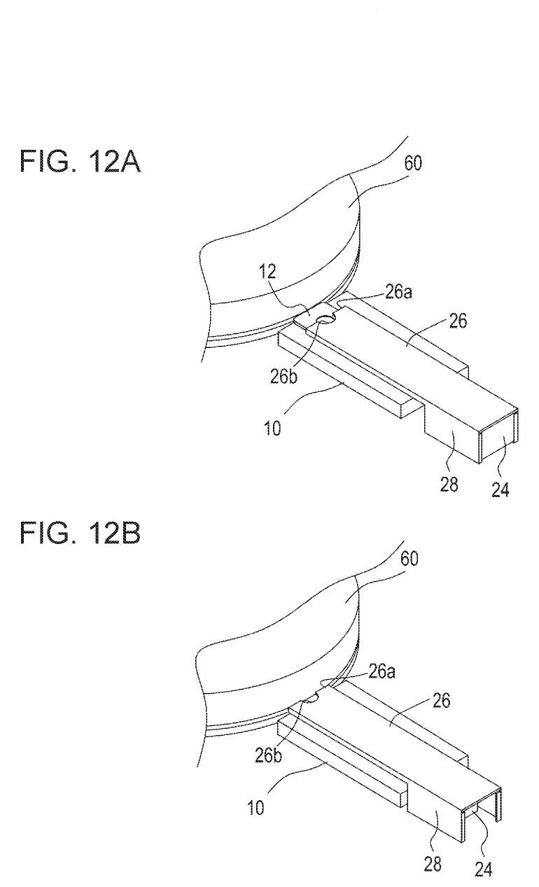

As illustrated in FIG. 12A, the analysis chip 12 on which a test substance sample has been spotted is further transported to the incubator 60. After the tip 26a of the cover member 26 comes into contact with the outer peripheral wall 65A of the rotating body 65 of the incubator 60 (see FIG. 9), the cover member 26 is retracted from the protruding position as illustrated in FIG. 12B, and only the pressing member 22 advances to thereby complete transportation of the analysis chip 12 to the cell S. After the analysis chip 12 is loaded in the cell S, the pressing member 22 and the cover member 26 are retracted rearward in the transportation direction.

The analysis chip 12 is heated to a target temperature (for example, 37°C) in the incubator 60 and is transported to a measurement position, and colorimetry measurement of the analysis chip 12 is performed by the optical measurement unit 70. The analysis chip 12 for which the colorimetry measurement has been performed is transported to a position at which the analysis chip 12 can be transported to a disposal position in the incubator 60 and is transported to the disposal position by a disposal transportation mechanism (not illustrated). The process of analyzing the test substance sample is thus completed.

As described above, the analysis apparatus 100 according to the present embodiment includes the transportation mechanism 20 including the plate-shaped pressing member 22 that presses the analysis chip 12 toward the target position by pressing the rear end 12b of the analysis chip 12 with the tip 22a, and the cover member 26 that can cover, in a state of protruding forward in the transportation direction beyond the tip 22a of the pressing member 22, a portion of the upper surface 12c of the analysis chip 12 on the side of the rear end 12b while the tip 22a of the pressing member 22 presses the rear end 12b of the analysis chip 12. With such a configuration, the analysis chip 12 can be transported in a state in which the rear end 12b and the upper surface 12c of the analysis chip 12 are in contact with the pressing member 22 and the cover member 26, respectively. Therefore, as compared with a case where the analysis chip 12 is pressed at a single portion, that is, the rear end 12b, the analysis chip 12 can be transported in a state in which the orientation of the analysis chip 12 is stabilized, and occurrence of malfunction during transportation can be suppressed.

In the analysis apparatus 100 in the present embodiment, as described above, the analysis chip 12 is transported in a state in which the rear end 12b and the upper surface 12c are in contact with the pressing member 22 and the cover member 26, respectively. Therefore, when the analysis chip 12 is transported to the spotting position P2 as the target position, the analysis chip 12 can be easily positioned at the spotting position P2.

In the present embodiment, since the cover member 26 includes the guide portion (here, the engagement portions 27) that positions the analysis chip 12 in the width direction, it is possible to suppress an inclination of the orientation of the analysis chip 12 relative to an advancing direction.

In the above-described embodiment, the engagement portions 27 each functioning as the guide portion are provided at two ends of the cover member 26. However, the guide portion that positions the analysis chip 12 in the width direction may be provided at the transportation table 10. For example, a projecting strip portion extending in the transportation direction may be provided as the guide portion at the transportation table 10.

In the present embodiment, the tip portion of the cover member 26 covering the upper surface 12c of the analysis chip 12 has the cutout for exposing the reaction region 13, which is a spotting portion of the analysis chip 12, and the cover member 26 covers the upper surface 12c of the analysis chip 12 in a state in which the reaction region 13 of the analysis chip 12 is exposed. With this configuration, a test substance can be spotted while the analysis chip 12 is held at the spotting position P2 in a state of being in contact with the pressing member 22 and the cover member 26, in particular, in a state of being urged against the transportation table 10 by the cover member 26. Without the need for the analysis apparatus 100 to include, at the spotting position P2, an additional mechanism for restricting the movement of the analysis chip 12, simplification of the configuration of the analysis apparatus 100 is addressed.

In the present embodiment, the cover member 26 is disposed so as to be slidable along the transportation direction relative to the pressing member 22. The transportation mechanism 20 has the urging member (here, the coil spring 29) that urges the cover member 26 toward the protruding position. When the cover member 26 comes into contact with the contact member (here, the outer peripheral wall 65A of the rotating body 65), an advance of the cover member 26 toward the target position is restricted, and the cover member 26 is retracted from the protruding position at which the cover member 26 protrudes forward beyond the tip 22a of the pressing member 22 in the transportation direction. Consequently, in a state in which the cover member 26 is in contact with the outer peripheral wall 65A and stops, it is possible to continue pressing of the rear end 12b of the analysis chip 12 with the tip 22a of the pressing member 22 toward the slit 65S provided in the outer peripheral wall 65A and possible to reliably transport the analysis chip 12 to the cell S, which is the target position, through the slit 65S.

In the above embodiment, a form of transporting the analysis chip 12 in a state of being placed at the standby position P1 on the transportation table 10 to the spotting position P2 and to the cell S has been described. However, the analysis chip 12 may be placed at the standby position P1 by any method. For example, a cartridge in which a plurality of stacked analysis chips are disposed may be installed adjacent to the standby position P1 on the transportation table 10, and the analysis chips may be transported one by one from the cartridge onto the transportation table 10 by a transportation bar or the like to be placed at the standby position P1.

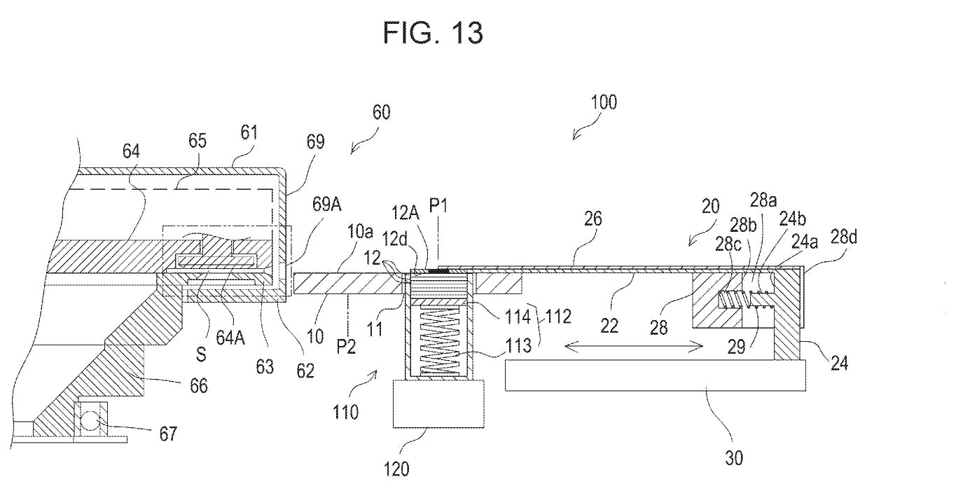

Further, as illustrated in FIG. 13, an opening 11 may be provided at the standby position P1 on the transportation table 10, and the analysis chip 12 may be supported in the opening 11 at a height at which a lower surface 12d of the analysis chip 12 coincides with a transportation surface 10a of the transportation table 10. In the example in FIG. 13, the analysis apparatus 100 includes a cartridge installation portion 120 at which a cartridge 110 that houses a plurality of the analysis chips 12 that are stacked is detachably installed. The cartridge installation portion 120 holds the cartridge 110 below the standby position P1 on the transportation table 10. The cartridge 110 includes a push-up mechanism 112 that pushes up, among the plurality of analysis chips 12 housed in the cartridge 110, an uppermost analysis chip 12A through the opening 11 to a height at which the lower surface 12d coincides with the transportation surface 10a of the transportation table 10.

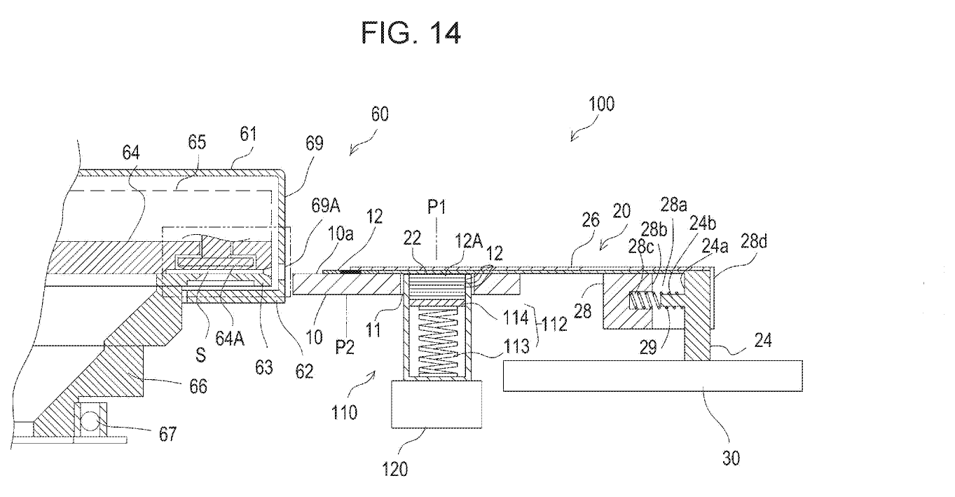

The push-up mechanism 112 includes a compression coil spring 113 and a support table 114. The compression coil spring 113 is included in a lower portion of an analysis-chip housing space. In the analysis-chip housing space, a plurality of the stacked analysis chips 12 are placed on the support table 114. The compression coil spring 113 is disposed on the lower surface side of the support table 114, and the support table 114 is urged upward by the compression coil spring 113. The cartridge 110 has an open top, and, in the present example, the pressing member 22 serves as a top plate of the cartridge 110 while the analysis chip 12 is transported from the standby position P1 toward the incubator 60 (see FIG. 14). As illustrated in FIG. 14, the pressing member 22 is configured to press the stacked analysis chips 12 from the side of the uppermost analysis chip 12A so that the analysis chip 12A does not come out of the cartridge 110. After the analysis chip 12 is transported to the incubator 60, when the pressing member 22 and the cover member 26 are retracted as illustrated in FIG. 13 until a step portion between the pressing member 22 and the cover member 26, which protrudes beyond the tip 22a of the pressing member 22, is positioned at the standby position P1, the pressing member 22 is positioned outside the standby position P1. Therefore, a gap is generated between the cover member 26 and the analysis chip 12A. The uppermost analysis chip 12A is pushed up into this gap, and the lower surface 12d of the analysis chip 12A becomes flush with the transportation surface 10a of the transportation table 10. Consequently, the analysis chip 12A is fitted to the stepped portion between the pressing member 22 and the cover member 26 and enters a transportable state. When the analysis chip 12A to be transported is held at the standby position P1 by the cover member 26 and the pressing member 22, the cover member 26 serves as the top plate of the cartridge 110. The method of transportation is the same as that in the previously described embodiment.

When such a cartridge installation portion 120 at which the cartridge 110 is detachably installed is included, it is possible to automatically and sequentially feed a plurality of the analysis chips 12 to the standby position P1 by loading the cartridge 110, and an additional transportation means for placing each analysis chip 12 at the standby position P1 is not required. Consequently, a reduction in the size of the analysis apparatus 100 and a reduction in costs for the analysis apparatus 100 can be addressed.

Regarding the above embodiments, the following appendixes are further disclosed.

### Appendix 1

An analysis apparatus in which an analysis chip having a flat plate shape and onto which a test substance sample is spotted is detachably loaded, the analysis apparatus being configured to analyze the test substance sample by using the analysis chip, the analysis apparatus including:

a transportation table on which the analysis chip is transported; and

a transportation mechanism that transports the analysis chip positioned at a standby position on the transportation table to a target position that is predetermined, while sliding the analysis chip on the transportation table by pressing a rear end of the analysis chip,

in which the transportation mechanism includes a pressing member and a cover member, the pressing member having a plate shape and being configured to press the analysis chip toward the target position by pressing the rear end of the analysis chip with a tip of the pressing member, the cover member being capable of covering, in a state of protruding forward in a transportation direction beyond the tip of the pressing member, a portion of an upper surface of the analysis chip on a side of the rear end while the tip of the pressing member presses the rear end of the analysis chip.

### Appendix 2

The analysis apparatus according to Appendix 1, in which at least one of the transportation table or the cover member includes a guide portion that positions the analysis chip in a width direction.

### Appendix 3

The analysis apparatus according to Appendix 1 or Appendix 2, including a loading unit provided with a cell in which the analysis chip is to be housed for measurement,

in which the target position is a position at which the cell is provided in the loading unit.

### Appendix 4

The analysis apparatus according to Appendix 3, in which the loading unit is an incubator that heats the analysis chip housed in the cell to a predetermined temperature.

Appendix 5

The analysis apparatus according to any one of Appendixes 1 to 4, in which the cover member is disposed to overlap the pressing member so as to be slidable along the transportation direction, and

in which the analysis apparatus further includes a contact member that comes into contact with the cover member at a predetermined position before the analysis chip reaches the target position, the contact member being configured to restrict an advance of the cover member to the target position and thereby retract the cover member from a protruding position at which the cover member protrudes forward in the transportation direction beyond the tip of the pressing member.

### Appendix 6

The analysis apparatus according to Appendix 5, in which the analysis apparatus has an urging member that urges the cover member toward the protruding position, and

in which the cover member retracts relative to the pressing member against an urging force of the urging member when coming into contact with the contact member and returns to the protruding position when separating from the contact member.

### Appendix 7

The analysis apparatus according to Appendix 5 or Appendix 6, including a loading unit provided with a cell in which the analysis chip is to be housed for measurement,

in which the target position is a position at which the cell is provided in the loading unit,

in which an opening frame having a slit through which the analysis chip passes is provided in a path for insertion into the cell, and

in which the contact member is the opening frame.

### Appendix 8

The analysis apparatus according to any one of Appendixes 1 to 7, including a spotting mechanism that spots the test substance sample onto the analysis chip,

in which the target position is a spotting position onto which the test substance sample is spotted.

### Appendix 9

The analysis apparatus according to Appendix 8, in which the analysis chip includes a spotting portion on a main surface of the analysis chip, and

in which the cover member covers the upper surface in a state in which the spotting portion of the analysis chip is exposed.

### Appendix 10

The analysis apparatus according to any one of Appendixes 1 to 9, in which the transportation table has an opening at the standby position,

in which the analysis apparatus includes a cartridge installation portion at which a cartridge that houses a plurality of the analysis chips that are stacked is detachably installed, the cartridge installation portion being configured to hold the cartridge below the standby position on the transportation table, and

in which the cartridge includes a push-up mechanism that pushes up, among the plurality of analysis chips housed in the cartridge, an uppermost analysis chip through the opening to a height at which a lower surface of the uppermost analysis chip coincides with a transportation surface of the transportation table.

The entire content of the disclosure of JP2023-140486 filed on August 30, 2023 is incorporated herein by reference.

All documents, patent applications, and technical standards described in the present specification are incorporated herein by reference to the same extent as if each document, each patent application, and each technical standard were specifically and individually indicated to be incorporated by reference.

What is claimed is:

1. An analysis apparatus in which an analysis chip having a flat plate shape and onto which a test substance sample is spotted is detachably loaded, the analysis apparatus being configured to analyze the test substance sample by using the analysis chip, the analysis apparatus comprising:
a transportation table on which the analysis chip is transported; and
a transportation mechanism that transports the analysis chip positioned at a standby position on the transportation table to a target position that is predetermined, while sliding the analysis chip on the transportation table by pressing a rear end of the analysis chip,
wherein the transportation mechanism includes a pressing member and a cover member, the pressing member having a plate shape and being configured to press the analysis chip toward the target position by pressing the rear end of the analysis chip with a tip of the pressing member, the cover member being capable of covering, in a state of protruding forward in a transportation direction beyond the tip of the pressing member, a portion of an upper surface of the analysis chip on a side of the rear end while the tip of the pressing member presses the rear end of the analysis chip.

2. The analysis apparatus according to claim 1, wherein at least one of the transportation table or the cover member includes a guide portion that positions the analysis chip in a width direction.

3. The analysis apparatus according to claim 1, comprising:
a loading unit provided with a cell in which the analysis chip is to be housed for measurement,
wherein the target position is a position at which the cell is provided in the loading unit.

4. The analysis apparatus according to claim 3, wherein the loading unit is an incubator that heats the analysis chip housed in the cell to a predetermined temperature.

5. The analysis apparatus according to claim 1,
wherein the cover member is disposed to overlap the pressing member so as to be slidable along the transportation direction, and
wherein the analysis apparatus further comprises a contact member that comes into contact with the cover member at a predetermined position before the analysis chip reaches the target position, the contact member being configured to restrict an advance of the cover member to the target position and thereby retract the cover member from a protruding position at which the cover member protrudes forward in the transportation direction beyond the tip of the pressing member.

6. The analysis apparatus according to claim 5,
wherein the analysis apparatus has an urging member that urges the cover member toward the protruding position, and
wherein the cover member retracts relative to the pressing member against an urging force of the urging member when coming into contact with the contact member and returns to the protruding position when separating from the contact member.

7. The analysis apparatus according to claim 5, comprising:
a loading unit provided with a cell in which the analysis chip is to be housed for measurement,
wherein the target position is a position at which the cell is provided in the loading unit,
wherein an opening frame having a slit through which the analysis chip passes is provided in a path for insertion into the cell, and
wherein the contact member is the opening frame.

8. The analysis apparatus according to claim 1, comprising:
a spotting mechanism that spots the test substance sample onto the analysis chip,
wherein the target position is a spotting position onto which the test substance sample is spotted.

9. The analysis apparatus according to claim 8,
wherein the analysis chip includes a spotting portion on a main surface of the analysis chip, and
wherein the cover member covers the upper surface in a state in which the spotting portion of the analysis chip is exposed.

10. The analysis apparatus according to claim 1,
wherein the transportation table has an opening at the standby position,
wherein the analysis apparatus comprises a cartridge installation portion at which a cartridge that houses a plurality of the analysis chips that are stacked is detachably installed, the cartridge installation portion being configured to hold the cartridge below the standby position on the transportation table, and
wherein the cartridge includes a push-up mechanism that pushes up, among the plurality of analysis chips housed in the cartridge, an uppermost analysis chip through the opening to a height at which a lower surface of the uppermost analysis chip coincides with a transportation surface of the transportation table.
